(19) United States
(12) Patent Application Publication
(10) Pub. No.: US 2025/0169904 A1
(43) Pub. Date: May 29, 2025

(54) SCALING OF SURGEON CONSOLE INPUTS FOR WRISTED ROBOTICALLY ASSISTED SURGICAL INSTRUMENTS

(71) Applicant: Covidien LP, Mansfield, MA (US)

(72) Inventor: Zachary S. Heiliger, Nederland, CO (US)

(21) Appl. No.: 18/838,679

(22) PCT Filed: Mar. 13, 2023
(86) PCT No.: PCT/IB2023/052402

§ 371 Date: Aug. 15, 2024

Related U.S. Application Data
(60) Provisional application No. 63/321,231, filed on Mar. 18, 2022.

(51) Int. Cl. A61B 34/37; A61B 34/00; A61B 90/00

(52) U.S. Cl. CPC A61B 34/37; A61B 34/74; A61B 34/77; A61B 90/06; A61B 2090/067

(57) ABSTRACT

A surgical robotic system includes a hand controller having a handle configured to rotate about at least one handle axis. The system also includes an instrument drive unit and an instrument coupled to the instrument drive unit. The instrument includes an end effector configured to rotate about at least one end effector axis. The system further includes a controller configured to receive an angle input based on rotation of the handle about the at least one handle axis. The controller is further configured to convert the angle input to an end effector angle using a conversion function and instruct the instrument drive unit to rotate the end effector about the at least one end effector axis to achieve the end effector angle.

### BACKGROUND

Surgical robotic systems may include a surgeon console controlling one or more surgical robotic arms, each including a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a work site within the patient's body. The surgeon console includes hand controllers which translate user input into movement of the surgical instrument and/or end effector.

### SUMMARY

Surgical robotic systems may operate with various types of wristed instruments, such as vessel sealers, graspers, dissectors, etc. While robotic instruments are constrained by design choices, i.e., limits on range of motion, human wrists are limited by anatomy. Thus, the range of motion of the robotic instrument may exceed the range of motion of human wrists. Accordingly, there is a need to adjust user input in view of anatomical limits range of motion of operator's wrists.

According to one embodiment of the present disclosure, a surgical robotic system is disclosed. The surgical robotic system includes a hand controller having a handle configured to rotate about at least one handle axis. The system also includes an instrument drive unit and an instrument coupled to the instrument drive unit. The instrument includes an end effector configured to rotate about at least one end effector axis. The system further includes a controller configured to receive an angle input based on rotation of the handle about the at least one handle axis. The controller is further configured to convert the angle input to an end effector angle using a conversion function and instruct the instrument drive unit to rotate the end effector about the at least one end effector axis to achieve the end effector angle.

Implementations of the above embodiment may include one or more of the following features. According to one aspect of the above embodiment, the conversion function may include multiplying the angle input by a linear enlargement conversion factor. The controller may be further configured to limit the angle input to an input rotational range. The controller may be further configured to determine the input rotational range and to calculate the linear enlargement conversion factor based on the determined input rotational range and the end effector angle. The input rotational range may be about 80 degrees. The end effector is configured to rotate within an end effector rotational range. The end effector rotational range may be larger than the input rotational range. The end effector rotational range may be about 120 degrees. The handle may be configured to rotate about a first handle axis and a second handle axis. The end effector may be configured to rotate about a pitch axis and a yaw axis. Rotation of the handle about the first handle axis may control the yaw axis of the end effector and rotation of the handle about the second handle axis may control the pitch axis of the end effector.

According to another embodiment of the present disclosure, a surgical robotic system is provided. The surgical robotic system includes a hand controller having a handle configured to rotate about at least one handle axis. The system also includes an instrument drive unit and an instrument coupled to the instrument drive unit. The instrument includes an end effector configured to rotate about at least one end effector axis within an end effector rotational range. The system also includes a controller configured to receive an angle input within an input rotational range based on rotation of the handle about the at least one handle axis, where the end effector rotational range is larger than the input rotational range. The controller is also configured to convert the angle input to an end effector angle using a conversion function and instruct the instrument drive unit to rotate the end effector about the at least one end effector axis to achieve the end effector angle.

Implementations of the above embodiment may include one or more of the following features. According to one aspect of the above embodiment, the handle may be configured to rotate about the at least one handle axis within a handle rotational range. The handle rotational range may be larger than the end effector rotational range. The conversion function may include multiplying the angle input by a linear enlargement conversion factor. The controller may be further configured to limit the angle input to the input rotational range. The input rotational range may be about 80 degrees. The end effector rotational range may be about 120 degrees.

According to a further embodiment of the present disclosure, a method for controlling a surgical robotic instrument is disclosed. The method includes receiving an angle input based on rotation of a handle about at least one handle axis and converting the angle input to an end effector angle using a conversion function. The method also includes instructing an instrument drive unit driving an instrument having an end effector to rotate the end effector about at least one end effector axis to achieve the end effector angle.

Implementations of the above embodiment may include one or more of the following features. According to one aspect of the above embodiment, converting the angle input may further include multiplying the angle input by a linear enlargement conversion factor. The method may also include limiting the angle input to an input rotational range that is smaller than an end effector rotational range.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a perspective view, with parts separated, of an instrument drive unit and a surgical instrument according to an embodiment of the present disclosure;

FIG. 6 is a top, perspective view of an end effector, according to an embodiment of the present disclosure, for use in the surgical robotic system of FIG. 1;

FIGS. 7A-C are perspective, schematic views of the end effector being moved to adjust yaw, pitch, and jaw angle, respectively, according to the present disclosure;

FIG. 8 is a perspective view of a hand controller of the surgeon console according to an embodiment of the present disclosure;

FIG. 9 is a perspective view of an end effector moved to a yaw limit according to an embodiment of the present disclosure;

FIG. 10 is a perspective view of the hand controller moved to a user input yaw limit corresponding to the end effector yaw limit of FIG. 9 according to an embodiment of the present disclosure;

FIG. 11 is a perspective view of an end effector moved to a pitch limit according to an embodiment of the present disclosure;

FIG. 12 is a perspective view of the hand controller moved to a user input pitch limit corresponding to the end effector pitch limit according to an embodiment of the present disclosure;

FIG. 13 is a flow chart of a method for scaling user input according to an embodiment of the present disclosure;

FIG. 14 shows a plot pitch and yaw angle of the end effector and corresponding pitch and yaw angle of the hand controller, according to an embodiment of the present disclosure; and

FIG. 15 shows a plot of a torque response and a superimposed plot of a force feedback response according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

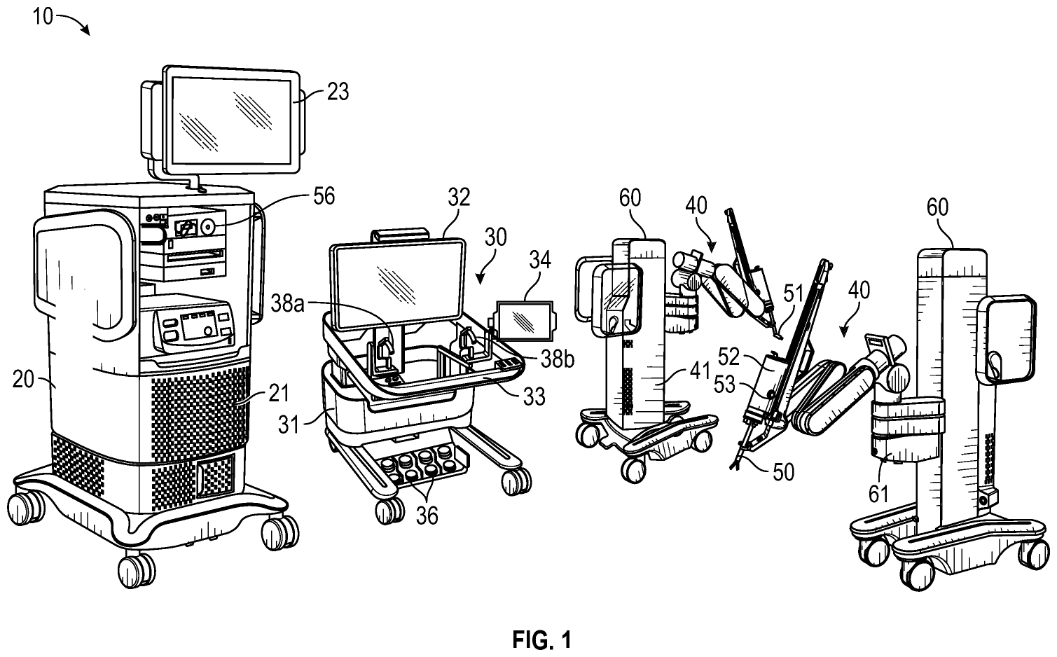

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 also couple to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

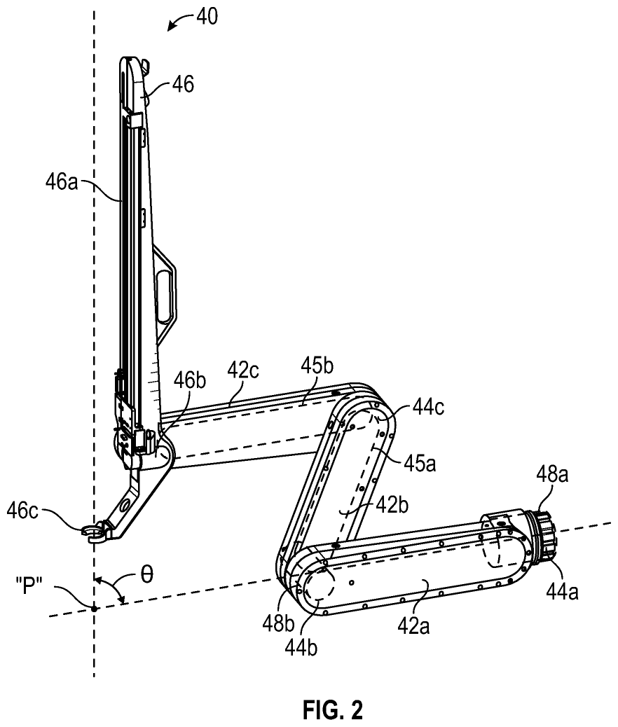

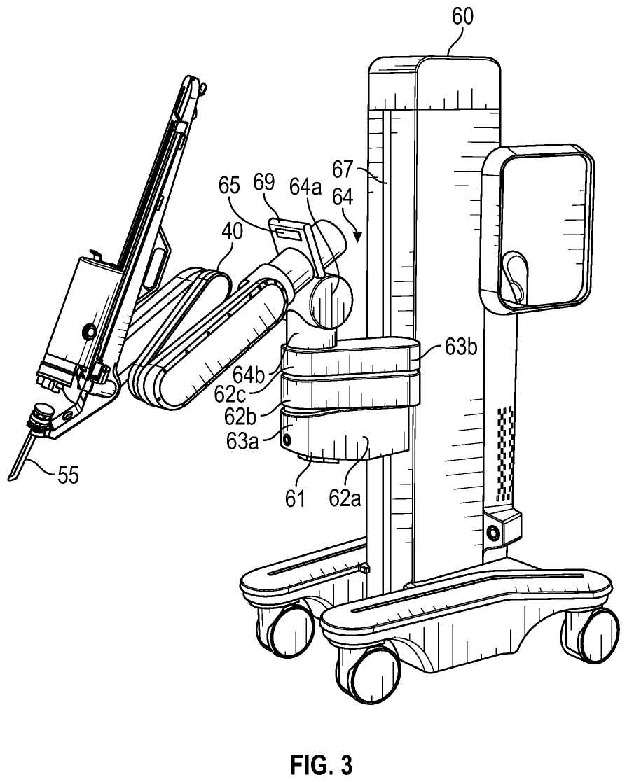

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

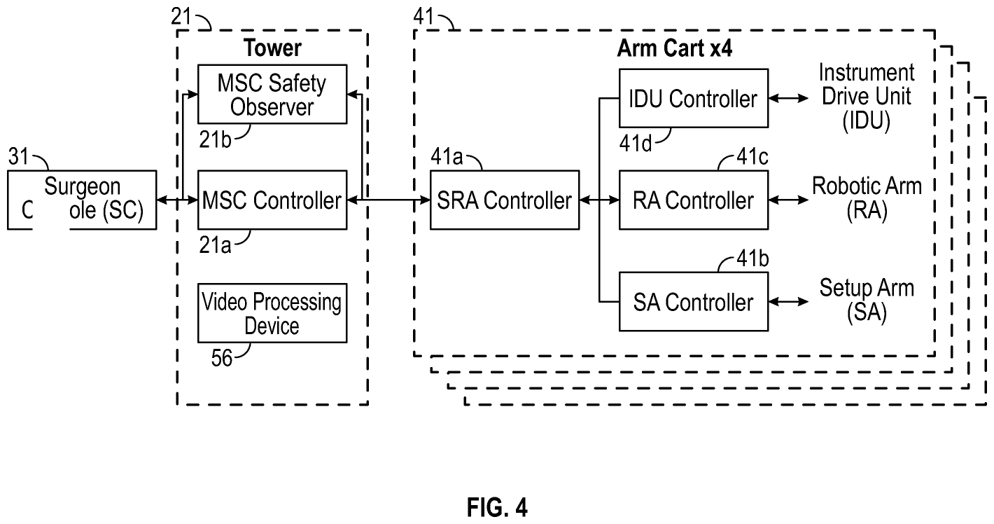

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

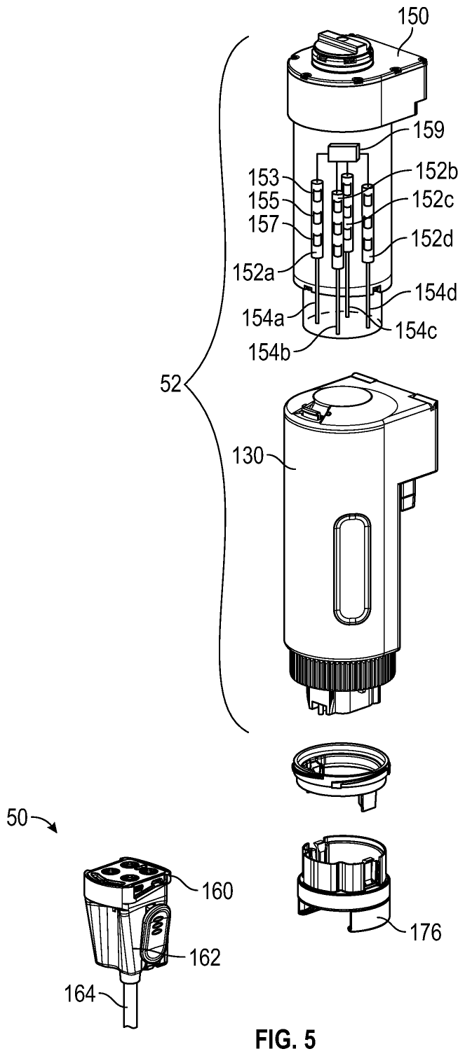

With reference to FIG. 5, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

The IDU 52 includes a motor pack 150 and a sterile barrier housing 130. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50.

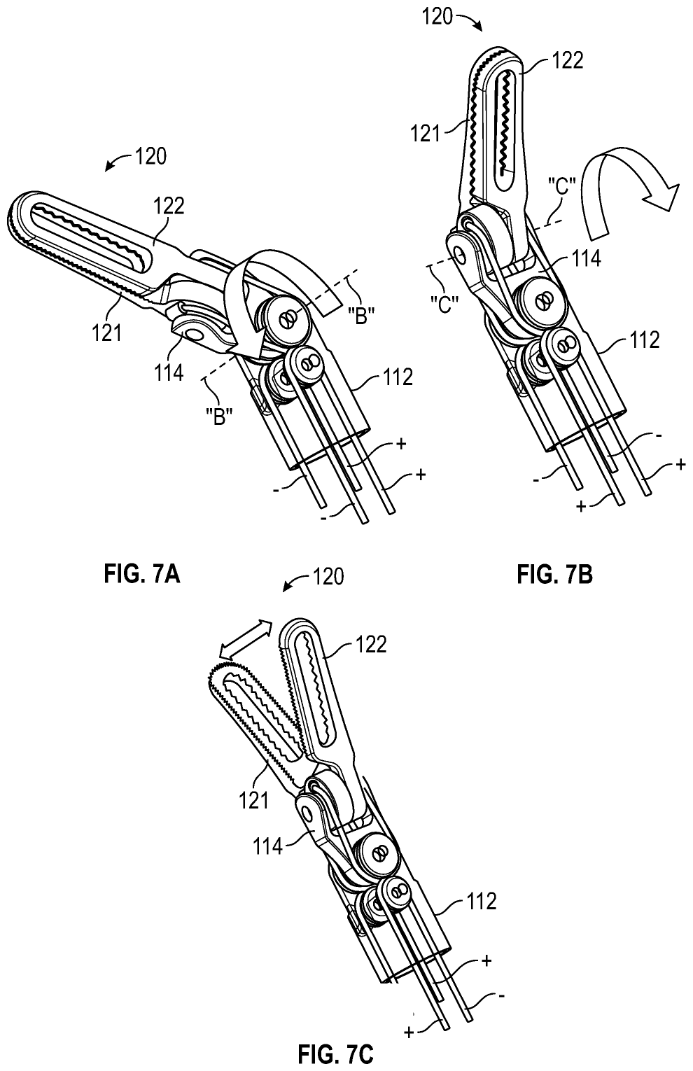

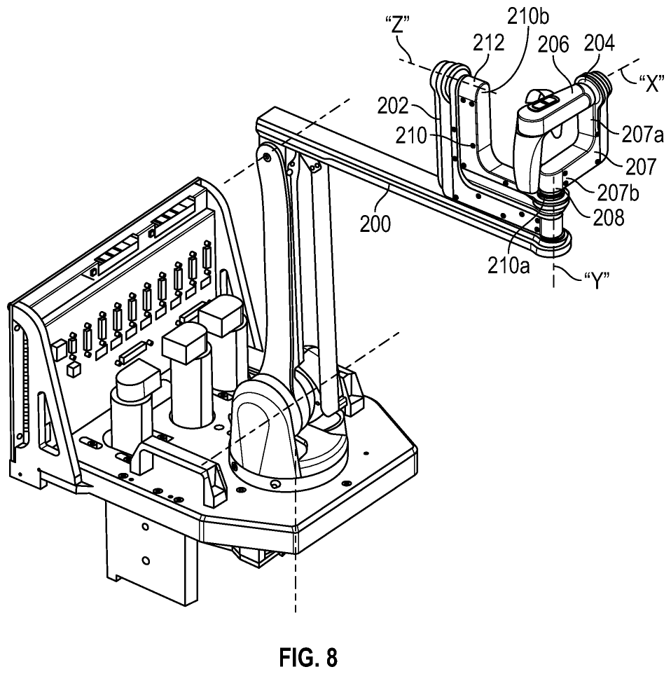

The instrument 50 is configured to transfer rotational forces/movement supplied by the IDU 52 (e.g., via the motors 152a-d of the motor pack 150) into longitudinal movement or translation of the cables or drive shafts to effect various functions of an end effector 120 (FIG. 7).

Each of the motors 152a-d includes a current sensor 153, a torque sensor 155, and an encoder sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder 157 may be any device that provides a sensor signal indicative of the number of rotations of the motor 152a, such as a mechanical encoder or an optical encoder. Parameters which are measured and/or determined by the encoder 157 may include speed, distance, revolutions per minute, position, and the like. The sensor signals from sensors 153, 155, 157 are transmitted to the IDU controller 41d, which then controls the motors 152a-d based on the sensor signals. In particular, the motors 152a-d are controlled by an actuator controller 159, which controls torque outputted and angular velocity of the motors 152a-d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With reference to FIG. 5, instrument 50 includes an adapter 160 having a housing 162 at a proximal end portion thereof and an elongated shaft 164 that extends distally from housing 162. Housing 162 of instrument 50 is configured to selectively couple to IDU 52 of robotic, to enable motors 152a-d of IDU 52 of robotic surgical assembly 100 to operate the end effector 120 of the instrument 50. Housing 162 of instrument 50 supports a drive assembly that mechanically and/or electrically cooperates with motors 152a-d of IDU 52 of robotic surgical assembly 100. Drive assembly of instrument 50 may include any suitable electrical and/or mechanical component to effectuate driving force/movement.

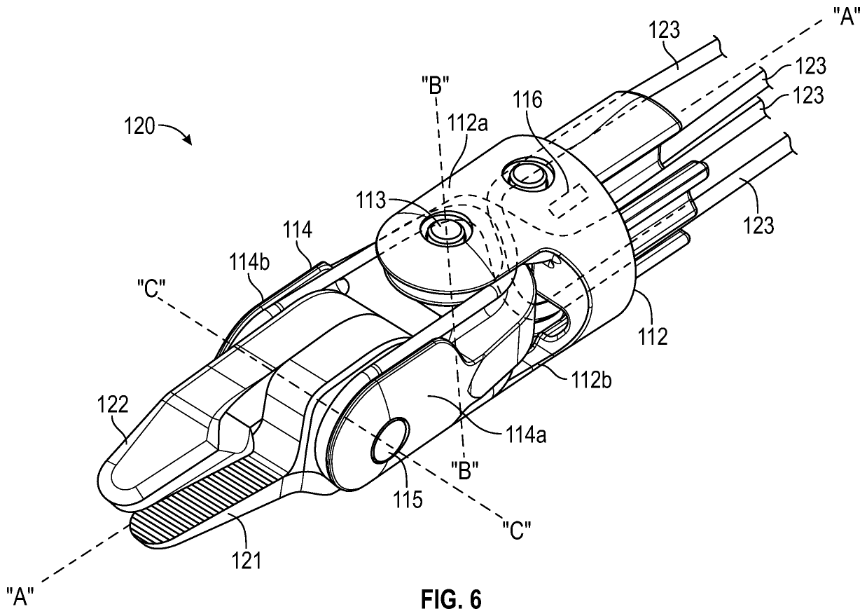

The surgical instrument also includes an end effector 120 coupled to the elongated shaft 164. The end effector 120 may include any number of degrees of freedom allowing the end effector 120 to articulate, pivot, etc., relative to the elongated shaft 164. The end effector 120 may be any suitable surgical end effector configured to treat tissue, such as a dissector, grasper, sealer, stapler, etc. As shown in FIG. 6, the end effector 120 may include a pair of opposing jaws 121 and 122 that are movable relative to each other.

In embodiments, the end effector 120 may include a proximal portion 112 having a first pin 113 and a distal portion 114. The end effector 120 may be actuated using a plurality of cables 123 routed through proximal and distal portions 112 and 114 around their respective pulleys 112a, 112b, 114a, 114b, which are integrally formed as arms of the proximal and distal portions 112 and 114. In embodiments, the end effector 120, namely, the distal portion 114 and the jaws 121 and 122, may be articulated about the axis “B-B” to control a yaw angle of the end effector with respect to a longitudinal axis “A-A” as shown in FIG. 7A. The distal portion 114 includes a second pin 115 with a pair of jaws 121 and 122 pivotably coupled to the second pin 115. The jaws 121 and 122 are configured to pivot about an axis “C-C” defined by the second pin 115 allowing for controlling a pitch angle of the jaws 121 and 122 as shown in FIG. 7B as well as opening and closing the jaws 121 and 122 as shown in FIG. 7C. The yaw, pitch, and jaw angles are controlled by adjusting the tension and/or length and direction (e.g., proximal or distal) of the cables 123. Thus, the end effector 120 may have three degrees of freedom, yaw, pitch, and jaw angle between jaws 121 and 122. The three degrees of freedom, i.e., yaw, pitch, and jaw angle, are manipulated by applying varying amounts of tension to four drive cables 123 of the instrument 50. Tension is applied to the drive cables 123 by four individually addressable motors 152a-d in the IDU 52.

The left- and right-hand controllers 38a and 38b are identical with the exception of the placement of a gripper paddle 201. Thus, the left-hand controller 38a is a mirror copy of the right-hand controller 38b and only the left-hand controller 38a is described in further detail below. Details of the hand controllers 38a and 38b are provided in U.S. Pat. No. 16/306,420, titled “Control arm assemblies for robotic surgical systems” filed on Nov. 30, 2018, and the entire contents of which are incorporated by reference herein.

The left-hand controller 38a includes a support arm 200 and a gimbal assembly 202 which is configured to receive rotational input for controlling the instrument 50 and/or the end effector 120. In particular, the gimbal assembly 202 includes a handle 206 coupled to a first end 207a of a first link 207 via a first rotation joint 204. The rotation joint 204 allows for rotation of the handle 206 about a first rotational axis, i.e., x-axis, defined by the first rotation joint 204. At a second end 207b, the first link 207 is also coupled to a first end 210a of a second link 210 via a second rotation joint 208. The second rotation joint 208 allows for rotation of the first link 207 about a second rotational axis, i.e., y-axis. A third rotation joint 212 is coupled to a second end 210b of the second link 210. The rotation joint 212 allows for rotation of the second link 210 about a third rotational axis, i.e., z-axis.

Rotation of the first, second, and third rotation joints 204, 208, and 212, may be measured using any suitable sensors (not shown), e.g., encoders, that provide rotational measurements to the controller 21a, or any other suitable controller of the robotic system 10. The controller 21a is configured to determine rotational movement of the gimbal assembly 202 based on rotational measurements from the sensors.

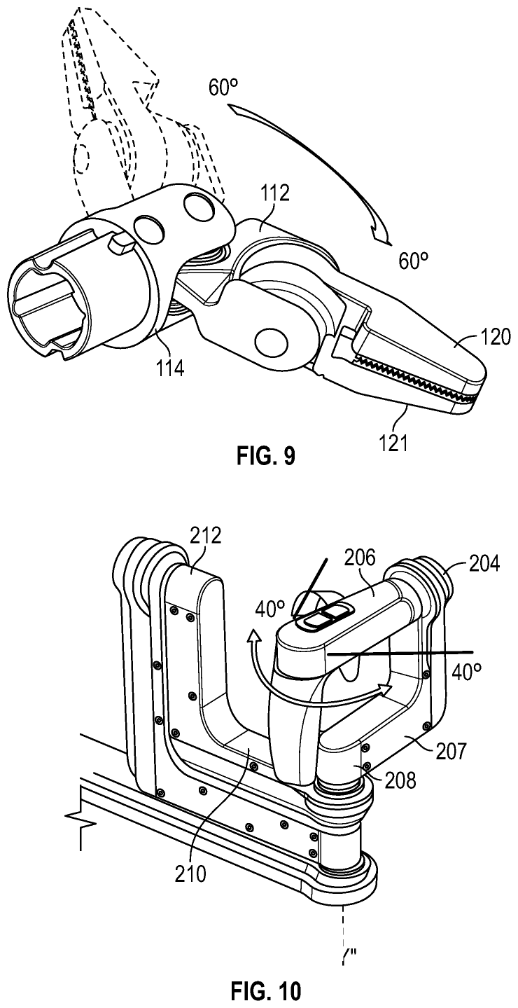

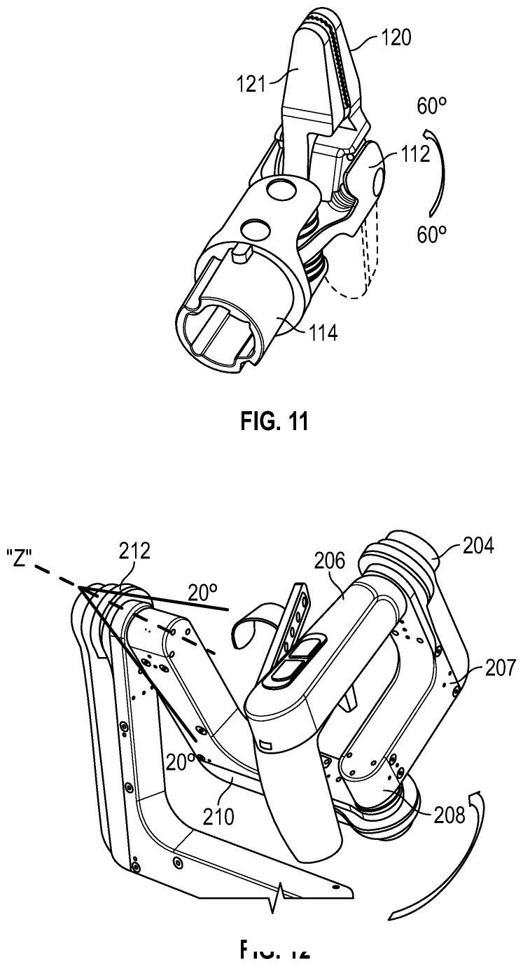

Since the hand controllers 38a and 38b are operated by gripping the handle 206, the movements of the handle 206 within the gimbal assembly 202 is limited by the anatomical limits of the human wrist during pronation, supination, flexion, extension, and deviation movements. Flexion and extension movements, i.e., bending of the hand at the wrist up or down respectively while the hand is in a horizontal plane, are used to rotate the handle 206 along with the first link 207 about the second rotational axis as shown in FIG. 11. Rotation of the handle 206 and the first link 207 about the second rotational axis may be used to control the yaw of the end effector 120, i.e., rotation about the “B-B” axis as shown in FIGS. 7A and 9.

Radial and ulnar deviations, i.e., bending of the hand at the wrist side-to-side while the hand in a horizontal plane, are used to rotate the handle 206 along with the first link 207 and the second link 210 about the third rotational axis as shown in FIG. 12. Rotation of the handle 206 as well as the first and second links 207 and 210 is used to control the pitch of the end effector 120, i.e., rotation about the “C-C” axis as shown in FIGS. 7B and 11. The wrist may be rotated relative to the forearm, i.e., pronated and supinated, which would rotate the handle 206 about the first rotation axis, which results in rotation of the end effector 120 about the longitudinal axis “A-A.”

The rotational range of the end effector 120 about the “B-B” and “C-C” axes may be from about 90 degrees to about 160 degrees, and in embodiments may be about 120 degrees. Rotational range of the end effector 120 about “A-A” axis may be from about 180 degrees to about 360 degrees. In contrast, each of the rotation joints 204, 208, 212 may allow for any range of rotation, e.g., about 180 degrees, which defines a handle or gimbal rotational range. In embodiments, the handle rotational range may allow for full 360 degrees of rotation about each of the axes of the gimbal assembly 202. Thus, the rotational ranges of the rotation joints 204, 208, 212 may exceed the rotational ranges of the human wrist operating the hand controllers 38a and 38b, which may only be from about 80 degrees for flexion and extension and about 40 degrees for ulnar and radial deviation. Accordingly, the rotational range of the end effector 120 may exceed the rotational range of the human wrist.

The present disclosure provides a control algorithm, which may be embodied as software instructions executed by a controller, e.g., the controller 21a or any other suitable controller of the system 10. The controller 21a is configured to process rotational inputs through the rotation joints 204, 208, 212 to control the instrument 50 and/or the end effector 120 to allow for movement of the end effector 120 to its maximum limit despite the movement limits of the human wrist. In contrast, a one-to-one control scheme, where each degree of rotation of the gimbal assembly 202 results in a degree of rotation of the end effector 120 would not utilize the full range of motion of the end effector 120 unless clutching, i.e., back and forth movements of the handle 206, or other input techniques was used. The controller 21a according to the present disclosure utilizes linear or non-linear scaling to match the range of motion of the human wrist to the end effector 120. In addition, the controller 21a is also configured to provide for proportional force feedback. The handle 206 includes a haptic device that is configured to vibrate or otherwise provide force feedback to the user grasping the handle 206.

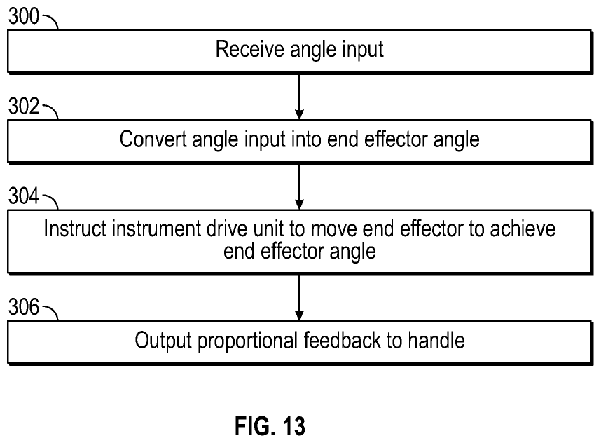

With reference to FIG. 13, a method of the control algorithm includes initially receiving an angle input at the controller 21a at step 300. Receiving angle input includes measuring the angle of rotation of any of the rotation joints 204, 208, 212 during movement of the handle 206 and/or the gimbal assembly 202. The angle input may also be limited based on the rotational range of rotation joints 204, 208, 212, which may exceed the limits of the human wrist. Thus, the angle input may be initially truncated if they exceed present movement limits, e.g., 80 degrees for yaw and pitch adjustments.

The controller 21a then converts the angle input into desired end effector angle at step 302. Conversion is based on the type of angle input, i.e., rotation about the first rotational axis rotates the end effector about the “A-A” axis, rotation about the second rotational axis rotates the end effector about the “B-B” axis, and rotation about the third rotational axis rotates the end effector about the “C-C” axis. Each of the angle input types is then converted using a conversion function having a linear or non-linear enlargement conversion factor.

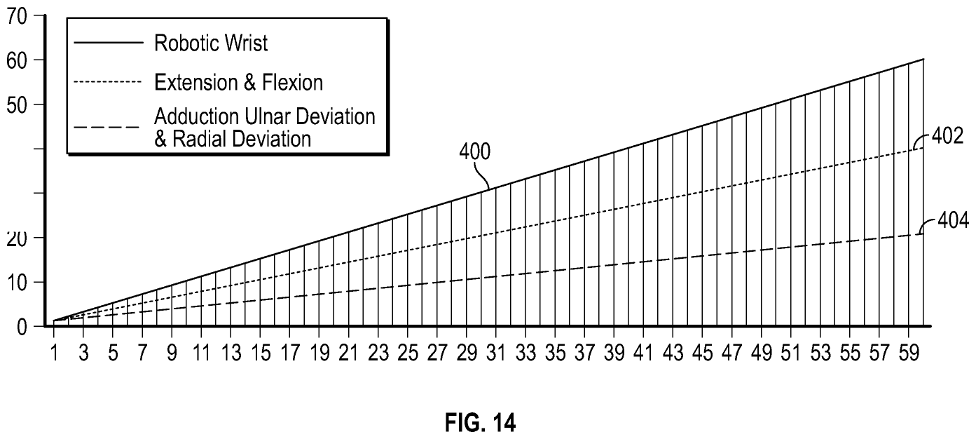

The controller 21a may execute a conversion function, e.g., multiplication, on the angle input using a conversion factor. FIG. 14 shows an angle plot 400 for pitch and yaw rotation of the end effector 120, an angle plot 402 for flexion and extension movement, and an angle plot 404 for radial and ulnar deviation. The plots 400, 402, 404 represent half of the rotational ranges, namely, from a 0 midpoint to one of the limits of the range. As noted above, flexion and extension movement are measured using the rotation joint 208 and deviation is measured using the rotation joint 212. Angle inputs through the rotation joints 208 and 212 may be limited by the controller 21a to a desired range corresponding to anatomical limits described above, e.g., about 40 degrees in either direction for flexion and extension and about 20 degrees in either direction for deviation.

The plots 400, 402, 404 illustrate the conversion factor that may be used to convert the input from the rotation joint 208 into yaw adjustment of the end effector and the rotation joint 212 into pitch adjustment. The conversion factor may be unique for each of the angle input types. In embodiments, the conversion factor for the angle input of the rotation joint 208 may be about 1.5 and the conversion factor for the angle input of the rotation joint 212 may be about 3. The conversion factor depends on the rotational range of the end effector 120 and input range and may be any real number that is used to convert, i.e., enlarge, the input angle to a desired angle of the end effector 120 in a linear manner. Thus, each received angle input value is multiplied by the conversion factor to calculate the end effector angle 120. At step 304, the controller 21a instructs the IDU 52 to adjust the motors 152a-d to achieve the calculated end effector angle 120.

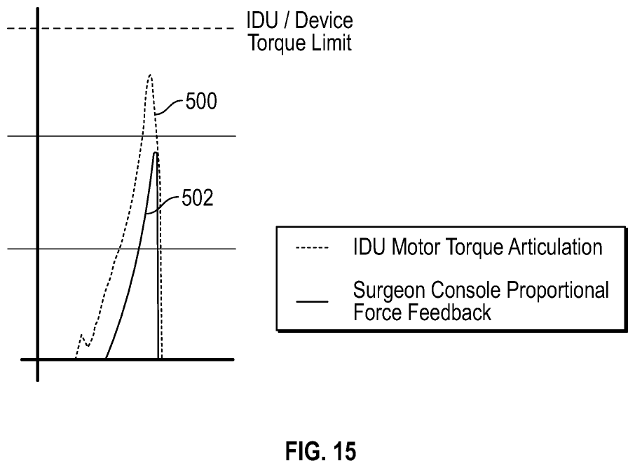

At step 306, the controller 21a calculates a feedback signal for activating a haptic device of the handle 206. As the IDU 52 is activating the motors 152a-d, the corresponding torque sensors 155 measure torque imparted on the motors 152a-d during movement. The torque signal is provided to the controller 21a, which then calculates the feedback signal in a linear manner as shown in FIG. 15. A torque plot 500 and a force feedback plot 502 are shown side-by-side to demonstrate that the controller 21a calculates the force feedback signal in a linear manner, similar to the linear conversion of the angle input.

In embodiments, the motion limits of the user's wrist may be determined prior to performing any scaling, e.g., during user setup of the surgeon console 30. The user may be requested to move each of the wrists on the handle controllers 38a and 38b through each of movements used in controlling the system 10, i.e., pronation, supination, flexion, extension, and deviation movements. The surgeon console 30 records and stores the limits and may then calculate an enlargement factor(s) for each of the movements. As described above, the factors may then be used to scale user input to achieve a desired pose for the instrument 50.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a hand controller including a handle configured to rotate about at least one handle axis;
an instrument drive unit;
an instrument coupled to the instrument drive unit, the instrument including an end effector configured to rotate about at least one end effector axis; and
a controller configured to:
receive an angle input based on rotation of the handle about the at least one handle axis;
convert the angle input to an end effector angle using a conversion function; and
instruct the instrument drive unit to rotate the end effector about the at least one end effector axis to achieve the end effector angle.

2. The surgical robotic system according to claim 1, wherein the conversion function includes multiplying the angle input by a linear enlargement conversion factor.

3. The surgical robotic system according to claim 2, wherein the controller is further configured to limit the angle input to an input rotational range.

4. The surgical robotic system according to claim 3, wherein the input rotational range is about 80 degrees.

5. The surgical robotic system according to claim 3, wherein the end effector is configured to rotate within an end effector rotational range.

6. The surgical robotic system according to claim 5, wherein the end effector rotational range is larger than the input rotational range.

7. The surgical robotic system according to claim 6, wherein the end effector rotational range is about 120 degrees.

8. The surgical robotic system according to claim 1, wherein the handle is configured to rotate about a first handle axis and a second handle axis.

9. The surgical robotic system according to claim 8, wherein the end effector is configured to rotate about a pitch axis and a yaw axis and rotation of the handle about the first handle axis controls the yaw axis of the end effector and rotation of the handle about the second handle axis controls the pitch axis of the end effector.

10. The surgical robotic system according to claim 3, wherein the controller is further configured to determine the input rotational range and to calculate the linear enlargement conversion factor based on the determined input rotational range and the end effector angle.

11. A surgical robotic system comprising:
a hand controller including a handle configured to rotate about at least one handle axis;
an instrument drive unit;
an instrument coupled to the instrument drive unit, the instrument including an end effector configured to rotate about at least one end effector axis within an end effector rotational range; and
a controller configured to:
receive an angle input within an input rotational range based on rotation of the handle about the at least one handle axis, wherein the end effector rotational range is larger than the input rotational range;
convert the angle input to an end effector angle using a conversion function; and
instruct the instrument drive unit to rotate the end effector about the at least one end effector axis to achieve the end effector angle.

12. The surgical robotic system according to claim 11, wherein the handle is configured to rotate about the at least one handle axis within a handle rotational range.

13. The surgical robotic system according to claim 12, wherein the handle rotational range is larger than the end effector rotational range.

14. The surgical robotic system according to claim 11, wherein the conversion function includes multiplying the angle input by a linear enlargement conversion factor.

15. The surgical robotic system according to claim 11, wherein the controller is further configured to limit the angle input to the input rotational range.

16. The surgical robotic system according to claim 11, wherein the input rotational range is about 80 degrees.

17. The surgical robotic system according to claim 11, wherein the end effector rotational range is about 120 degrees.

18. A method for controlling a surgical robotic instrument, the method comprising:
receiving an angle input based on rotation of a handle about at least one handle axis;
converting the angle input to an end effector angle using a conversion function; and
instructing an instrument drive unit driving an instrument having an end effector to rotate the end effector about at least one end effector axis to achieve the end effector angle.

19. The method according to claim 18, wherein converting the angle input further includes multiplying the angle input by an enlargement conversion factor.

20. The method according to claim 18, further comprising:
limiting the angle input to an input rotational range that is smaller than an end effector rotational range.
